(19) United States
(12) Patent Application Publication
(10) Pub. No.: US 2024/0138758 A1
(43) Pub. Date: May 2, 2024

(54) SYSTEMS AND METHODS FOR DETERMINING SLEEP STAGE AND A SLEEP QUALITY METRIC

(71) Applicant: The Alfred E. Mann Foundation for Scientific Research, Valencia, CA (US)

(72) Inventors: Brian V. MECH, Buffalo, MN (US); Sahar ELYAHOODAYAN, Los Angeles, CA (US); Hemang TRIVEDI, San Jose, CA (US)

(21) Appl. No.: 18/495,620

(22) Filed: Oct. 26, 2023

Related U.S. Application Data
(60) Provisional application No. 63/381,042, filed on Oct. 26, 2022.

(51) Int. Cl. A61B 5/00; A61B 5/0205; G16H 40/67; G16H 50/30

(52) U.S. Cl. CPC A61B 5/4815; A61B 5/0015; A61B 5/0205; A61B 5/4818; A61B 5/4836; A61B 5/7267; G16H 40/67; G16H 50/30; A61N 1/3601

(57) ABSTRACT

The present disclosure generally relates to systems and methods for determining and/or monitoring a sleep stage and/or a sleep quality metric for an individual using one or more sensors, and methods of treating medical conditions related thereto (e.g., obstructive sleep apnea).

### CROSS-REFERENCE TO RELATED APPLICATION

The application claims the benefit of U.S. Provisional Application No. 63/381,042, filed Oct. 26, 2022, which is herein incorporated by reference.

### TECHNICAL FIELD

The present disclosure generally relates to systems and methods for determining and/or monitoring the sleep stage and sleep quality of an individual using one or more sensors, and methods of treating medical conditions related thereto.

### BACKGROUND

Obstructive Sleep Apnea (“OSA”) is a sleep disorder involving obstruction of the upper airway during sleep. The obstruction of the upper airway may be caused by the collapse of or increase in the resistance of the pharyngeal airway, often resulting from tongue obstruction. The obstruction of the upper airway may be caused by reduced genioglossus muscle activity during the deeper states of NREM sleep. Obstruction of the upper airway may cause breathing to pause during sleep. Cessation of breathing may cause a decrease in the blood oxygen saturation level, which may eventually be corrected when the person wakes up and resumes breathing. The long-term effects of OSA include high blood pressure, heart failure, strokes, diabetes, headaches, and general daytime sleepiness and memory loss, among other symptoms.

OSA is extremely common and may have a prevalence similar to diabetes or asthma. Over 100 million people worldwide suffer from OSA, with about 25% of those people being treated. Continuous Positive Airway Pressure (“CPAP”) is a conventional therapy for people who suffer from OSA. More than five million patients own a CPAP machine in North America, but many do not comply with use of these machines because they cover the mouth and nose and, hence, are cumbersome and uncomfortable.

Neurostimulators may be used to open the upper airway as a treatment for alleviating apneic events. Such therapy may involve stimulating the nerve fascicles of the hypoglossal nerve (“HGN”) that innervate the intrinsic and extrinsic muscles of the tongue in a manner that prevents retraction of the tongue which would otherwise close the upper airway during the inspiration period of the respiratory cycle. For example, current stimulator systems may be used to stimulate the trunk of the HGN with a nerve cuff electrode. However, some of these systems do not provide a sensor or sensing capabilities, and therefore, the stimulation delivered to the HGN trunk is not synchronized to the respiratory cycle or modulated based upon the wakefulness of the individual being treated.

### BRIEF SUMMARY

Ideally, a system for treating OSA should account for the sleep stage of the individual being treated. Stimulation only needs to be applied when the individual is asleep, and may only need to be applied during certain sleep stages. Accordingly, a system that accounts for wakefulness may improve battery life by detecting or monitoring wakefulness and then adjusting one or more parameters in response (e.g., one or more sensors may be disabled or switched to a low-power mode when a patient is determined to be awake). Moreover, a system designed to account for wakefulness would be less likely to incorrectly apply stimulation (e.g., a system that accounts for the position or movement of an individual, but not wakefulness, may incorrectly apply stimulation to a patient laying in a supine state while awake). To date, current OSA stimulation systems have failed to provide this functionality, resulting in a need in the art for improved OSA stimulation systems that account for wakefulness as a parameter when selecting or applying stimulation, and in connection with the operation of the system generally.

Moreover, there is a need in the art for OSA stimulation systems that can evaluate a user's sleep quality, e.g., by tracking the amount of time that the user spends in sleep stages N1, N2, N3, and REM sleep. A sleep quality metric may be calculated based on sleep stage data and can provide feedback regarding the efficacy of an OSA treatment regimen. A physician or other medical professional may consider a subject's sleep quality metric when selecting stimulation parameters. For example, if a subject is found to spend little time in deep sleep, or multiple sleeping disturbances are detected, the OSA stimulation may need to be titrated to a higher level. Unfortunately, current OSA stimulation systems fail to provide this functionality, and the equipment needed to accurately track how much time a subject spends in each sleep stage is impractical for use outside of a polysomnography (“PSG”) study.

The present disclosure addresses these and other shortcomings by providing OSA stimulation systems that can accurately detect and/or monitor a subject's sleep stage (e.g., to generate one or more sleep quality metrics) using one or more sensors incorporated into or in communication with the system. Such systems may advantageously display improved power efficiency, accuracy, and/or functionality compared to current systems, among other benefits which will become apparent in view of the following description and the accompanying figures. For example, the present disclosure provides systems and methods that more accurately determine sleep stage with an implant, derive a sleep score, and use that score and other data to inform patients, and providers about sleep, motivate patients, and potentially allow clinicians to adjust implant settings in person, or remotely, to improve sleep quality, and to gauge the effectiveness of an implant. Determining how much time a person spends in each of the four standard sleep stages in a given night is a key marker of their sleep quality. While several factors influence the amount of time spent in each stage, to date, only electroencephalography (“EEG”) is capable of precisely determining which stage a person is in. Devices capable of accurately recording EEG are bulky and not typically conducive to comfortable sleep and hence are reserved for formal PSG studies. While wearable devices using different sensors have developed algorithms to estimate sleep stages and sleep quality (in the absence of EEG data), it is possible to do this more accurately and less obtrusively with an implanted device.

In a first general aspect, the disclosure provides a computer-implemented system for determining a sleep stage and/or sleep quality metric for a human subject, comprising: one or more sensors, wherein each sensor is configured to collect sensor data indicative of respiratory activity and/or a physical state of the human subject when placed on, in proximity to, or implanted in, the human subject; and a controller comprising a processor and memory, communicatively linked to the one or more sensors and configured to receive the sensor data from the one or more sensors, and determine the sleep stage and/or sleep quality metric for the human subject, using the received sensor data, wherein the controller is configured to perform the determination using a trained classifier which comprises an electronic representation of a classification system.

In some aspects, the one or more sensors each comprise: a pressure sensor, an accelerometer, a gyroscope, an auscultation sensor, a heart rate monitor, an electrocardiogram (“ECG”) sensor, a blood pressure sensor, a blood oxygen level sensor, an electromyography (“EMG”) sensor, and/or a muscle sympathetic nerve activity (“MSNA”) sensor. In some aspects, each sensor is independently positioned on, in proximity to, or as an implant within, the human subject.

In some aspects, the controller is further configured to receive biomarker data for the human subject comprising a concentration or amount of one or more biomarkers of the human subject, and to use this biomarker data when determining the sleep stage and/or sleep quality metric for the human subject. In some aspects, the one or more biomarkers comprise a concentration or amount of epinephrine, norepinephrine, cortisol, melatonin, serotonin, glucose, insulin, dopamine, noradrenaline, 5-hydroxoindiolacytic acid, glutamate, blood alcohol, tryptophan, kynurenine, and/or one or more inflammatory cytokines, in the human subject's blood or tissue.

In some aspects, the controller is configured to determine the sleep stage and/or sleep quality metric for the human subject using a) sensor data received from at least or exactly 1, 2, 3, 4, 5, 6, 7, 8, 9, or 10 sensors; and/or b) biomarker data comprising a concentration or amount of at least or exactly 1, 2, 3, 4, 5, 6, 7, 8, 9, or 10 biomarkers.

In some aspects, the trained classifier was trained using a baseline dataset, wherein the baseline dataset comprises: a) data generated during a prior single or multi-night polysomnography (PSG) study of the human subject; and/or b) data generated from a prior single or multi-night PSG study of a population of human subjects. In some aspects, the baseline dataset comprises: a) sensor data from one or more sensors, where each sensor comprise: a pressure sensor, an accelerometer, a gyroscope, an auscultation sensor, a heart rate monitor, an ECG sensor, a blood pressure sensor, a blood oxygen level sensor, an EMG sensor, and/or an MSNA sensor; and/or b) concentration or amounts of one or more biomarkers comprise epinephrine, norepinephrine, cortisol, melatonin, serotonin, glucose, insulin, dopamine, noradrenaline, 5-hydroxoindiolacytic acid, glutamate, blood alcohol, tryptophan, kynurenine, and/or one or more inflammatory cytokines

In some aspects, the classifier comprises a machine learning and/or deep learning algorithm. In some aspects, the classifier comprises one or more of: AdaBoost, Artificial Neural Network (ANN) learning algorithm, Bayesian belief networks, Bayesian classifiers, Bayesian neural networks, Boosted trees, case-based reasoning, classification trees, Convolutional Neural Networks, decisions trees, Deep Learning, elastic nets, Fully Convolutional Networks (FCN), genetic algorithms, gradient boosting trees, k-nearest neighbor classifiers, LASSO, Linear Classifiers, naive Bayes classifiers, neural nets, penalized logistic regression, Random Forests, ridge regression, support vector machines, or an ensemble thereof,

In some aspects, the one or more sensors configured to collect sensor data indicative of respiratory activity and/or a physical state of the human subject does not include an electroencephalography (“EEG”) sensor

In some aspects, the sleep stage for the human subject is determined to be a sleep stage selected from awake, or N1, N2, N3, or REM sleep. In some aspects, the sleep quality metric for the human subject is determined to be a numeric score. In some aspects, the system is configured to output the determined sleep stage and/or sleep quality metric to a graphical or text-based interface of an electronic device. In some aspects, the electronic device is a discrete controller of the system, a computer, a smart phone, a tablet, or a wearable device

In a second general aspect, the disclosure provides a method for determining a sleep stage and/or sleep quality metric for a human subject comprising: collecting sensor data indicative of respiratory activity and/or a physical state of the human subject, using one or more sensors configured to collect data when placed on, in proximity to, or implanted in, the human subject; receiving, by a controller comprising a processor and memory, the sensor data from the one or more sensors; determining the sleep stage and/or sleep quality metric for the human subject, using the received sensor data; wherein the controller is configured to perform the determination using a trained classifier comprising an electronic representation of a classification system, and/or to transmit the received sensor data to a server configured to perform the determination using a trained classifier comprising an electronic representation of a classification system.

In a third general aspect, the disclosure provides a method for determining a sleep stage and/or sleep quality metric for a human subject comprising: providing a system according to any of the exemplary aspects described herein, and determining the sleep stage and/or sleep quality metric for the human subject, using the provided system.

In a fourth general aspect, the disclosure provides a computer-implemented system for determining a sleep stage and/or sleep quality metric for a human subject, comprising: one or more sensors, wherein each sensor is configured to collect sensor data indicative of respiratory activity and/or a physical state of the human subject when placed on, in proximity to, or implanted in, the human subject; and a controller comprising a processor and memory, communicatively linked to the one or more sensors and configured to receive the sensor data from the one or more sensors, and transmit data based on the received sensor data to at least one local, remote, or cloud-based server, wherein the at least one local, remote, or cloud-based server is configured to determine a sleep quality metric for the human subject using a trained classifier configured to process the transmitted data.

In some aspects, the controller is further configured to transmit biomarker data for the human subject, comprising a concentration or amount of one or more biomarkers, to the at least one local, remote, or cloud-based server; and the at least one local, remote, or cloud-based server is configured to use the transmitted biomarker data when determining the sleep quality metric for the human subject using the trained classifier.

In a fifth general aspect, the disclosure provides a system for treating obstructive sleep apnea, comprising: the system for determining a sleep stage and/or sleep quality metric for a human subject, according to any one of the exemplary aspects described herein, and a stimulation system, communicatively linked to the controller and configured to deliver stimulation to a nerve which innervates an upper airway muscle of the human subject based on the sleep stage and/or sleep quality metric of the human subject determined by the controller.

In some aspects, the controller is configured to cause the stimulation system to apply, increase, decrease, temporarily pause, or terminate the stimulation based on the sleep stage and/or sleep quality metric of the human subject. In some aspects, the controller is configured to cause the stimulation system to change an amplitude, pulse width, or frequency of the stimulation based on the sleep stage and/or sleep quality metric of the human subject.

In a sixth general aspect, the disclosure provides methods of treating sleep apnea using the system according to any of the exemplary aspects described herein.

It is understood that any of the systems described herein may be configured to store, output, and/or transmit any of the data or parameters described herein. For example, the system may be configured to store actual and/or mean respiratory interval data for the human subject, and/or to output or transmit it to another local or remote device (e.g., a tablet computer or a discrete external controller communicatively linked with the system). In some aspects, the system may be configured to transmit such data to an external server or other local or remote storage (e.g., to archive such data or to provide it to a medical professional for further review). Accordingly, the systems described herein may incorporate a wired or wireless communication means (e.g., Bluetooth or wireless connectivity). The systems described herein may further be configured to allow a user, medical professional, or other party to modify one or more parameters of the system (e.g., the threshold parameter used to determine wakefulness level or sleep stage may be configurable by a medical professional). Updated parameters may be entered manually (e.g., using a dedicated external controller or paired computer or tablet) or received, e.g., as an updated configuration file provided wirelessly from a remote user.

To the accomplishment of the foregoing and related ends, the one or more aspects comprise the features hereinafter fully described and particularly pointed out in the claims. The following description and the annexed drawings set forth in detail certain illustrative features of the one or more aspects. These features are indicative, however, of but a few of the various ways in which the principles of various aspects may be employed, and this description is intended to include all such aspects and their equivalents.

### BRIEF DESCRIPTION OF THE DRAWINGS

FIG. 1 is a diagram illustrating an exemplary embodiment of a system for treating OSA using a sleep stage and/or sleep quality metric determined for a human subject based upon sensor data. In this example, the system includes several external sensors (S1-S5) and an implanted sensor (S6) integrated into the house of an implanted OSA stimulation system.

FIG. 2 is a diagram illustrating another exemplary system according to the disclosure. In this example, data collected from a plurality of implanted and external sensors is collected and sent to a cloud-based platform for processing.

FIG. 3 is a conceptual flow diagram summarizing a method for determining a subject's sleep stage and a sleep quality metric using the systems described herein, and optionally the use of such systems to treat the subject for OSA. This example illustrates the use of alternative embodiments which allow for local or remote processing of sensor data collected from a subject and/or the sleep stage and quality determination for the subject.

FIG. 4 is a conceptual flow diagram summarizing a method for determining a subject's sleep stage and a sleep quality metric using the systems described herein, and optionally the use of such systems to treat the subject for OSA. In this example, the system is shown to be capable of incorporating the use of biomarker data as part of the determination process.

FIG. 5 is an exemplary respiratory waveform illustrating a period of abnormal respiratory activity followed by a period of normal respiratory activity.

### DETAILED DESCRIPTION

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

Several aspects of exemplary embodiments according to the present disclosure will now be presented with reference to various systems and methods. These systems and methods will be described in the following detailed description and illustrated in the accompanying drawings by various blocks, components, circuits, processes, algorithms, etc. (collectively referred to as “elements”). These elements may be implemented using electronic hardware, computer software, or any combination thereof. Whether such elements are implemented as hardware or software depends upon the particular application and design constraints imposed on the overall system.

By way of example, an element, or any portion of an element, or any combination of elements may be implemented as a “processing system” that includes one or more processors. Examples of processors include microprocessors, microcontrollers, graphics processing units (GPUs), central processing units (CPUs), application processors, digital signal processors (DSPs), reduced instruction set computing (RISC) processors, systems on a chip (SoC), baseband processors, field programmable gate arrays (FPGAs), programmable logic devices (PLDs), application-specific integrated circuits (ASICs), state machines, gated logic, discrete hardware circuits, and other suitable hardware configured to perform the various functionality described throughout this disclosure. One or more processors in the processing system may execute software. Software shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise.

Accordingly, in one or more exemplary embodiments, the functions described may be implemented in hardware, software, or any combination thereof. If implemented in software, the functions may be stored on or encoded as one or more instructions or code on a computer-readable medium. Computer-readable media includes computer storage media. Storage media may be any available media that can be accessed by a computer. By way of example, and not limitation, such computer-readable media can comprise a random-access memory (“RAM”), a read-only memory (ROM), an electrically erasable programmable ROM (“EEPROM”), optical disk storage, magnetic disk storage, other magnetic storage devices, combinations of the aforementioned types of computer-readable media, or any other medium that can be used to store computer executable code in the form of instructions or data structures that can be accessed by a computer.

As noted above, the present disclosure is generally directed to systems and methods for determining and/or monitoring the sleep stage and sleep quality of an individual using one or more sensors, and methods of treating medical conditions related thereto. Sleep, and more specifically quality of sleep, is now recognized as critical to human health. Lack of sleep, or poor sleep contributes significantly not only to poor cognitive performance, but also to a host of human health conditions including hypertension, heart failure, cognitive disorders, diabetes, and many others.

Along with stress, breathing disorders are the most common cause of compromised sleep, and among sleep related breathing disorders, sleep apnea is the most common. When a person is suspected of having a sleep related problem including sleep related breathing disorders (SBDs) they typically undergo a PSG study in a sleep lab. PSG studies measure eight key parameters during a course of a night's sleep: airflow, breathing rate and effort, blood oxygen level, body position, eye movement, muscle electrical activity, heart rate, and brain activity. Among these parameters, brain activity is measured using EEG which can accurately determine the amount of time spent in each of the 4 sleep stages (N1, N2, N3, and REM sleep). This EEG data coupled with wakefulness (a parameter based on the number and duration of awakenings) provides the most accurate assessment of sleep quality. However, clinical EEG systems use twenty-five or more electrodes distributed on the scalp, precisely located and often affixed with a viscous conductive material by highly trained personnel, to perform their measurements. As such, EEG systems are not conducive to routine use at home, not only because EEG studies are difficult to administer, but also because most people would not have difficulty sleeping in view of the complex wiring and equipment required by EEG systems.

In the last decade or so, a plethora of activity trackers, fitness trackers, and sleep trackers have been developed which are typically wearable devices (e.g., wrist-worn devices) that use sensors to detect actigraphy, heart rate, respiration rate, and/or blood oxygen levels and also impute physiological biomarkers like heart rate variability, body position, number of minutes active, number of steps taken, respiration rate, and even time spent in various sleep stages and hence a sleep score indicative of the quality of sleep. Recent estimates of the accuracy of the imputed time spent in each sleep stage vary between 50 and 80%. Accordingly, there is a need for more accurate sleep stage tracking. Systems according to the present disclosure may be used, among other things, to improve the accuracy of sleep stage determinations, particularly in individuals that happen to have an implantable device (e.g., an OSA stimulation system).

To that end, a system according to the disclosure may comprise an OSA stimulation system that includes one or more sensors for determining and/or monitoring the sleep stage and sleep quality of an individual. In some aspects, the one or more sensors may comprise a sensor incorporated into an implanted device (e.g., within the same housing that contains one or more components of an OSA stimulation system or other active implantable device). Data collected from an implanted sensor may be used to determine and/or monitor the sleep stage and sleep quality of an individual, alone or in combination with data collected from one or more external sensors.

In some aspects, the system may include one or more sensors (e.g., implanted within the subject) which are configured to collect sensor data comprising a variety of parameters, e.g., respiration, respiration cycle, respiration rate, breathing effort, airflow, activity, and body position of a subject being treated. These sensors may include pressure sensors, accelerometers, gyroscopes, auscultation sensors, and other means for collecting data regarding the physiological state or biomarkers of a subject. In some aspects, potential sensors may include a heart rate monitor, an EKG, a blood pressure sensor, a blood oxygen level sensor, an EMG, and/or an MSNA. Sensors located in the body are typically more accurate than those located outside the body, and so, to the extent that any of these measured parameters are indicative of sleep stage and/or quality, the implanted device has the ability to determine more information, and more accurate information than an external device.

Systems according to the disclosure may also be configured to account for the concentration or amount of one or more biomarkers of the individual being treated, including but not limited to epinephrine, norepinephrine, cortisol, melatonin, serotonin, glucose, insulin, dopamine, noradrenaline, 5-hydroxoindiolacytic acid, glutamate, blood alcohol, tryptophan, kynurenine, inflammatory cytokines, etc. Biomarker concentrations or amounts may be determined in real-time (e.g., using an implanted sensor). Alternatively, the concentration or amount of a biomarker may be assayed using a kit or external device (e.g., a glucose sensor) to provide a recent reading. It is understood that biomarker concentrations and amounts may be determined by an end user of a system according to the disclosure or by a medical professional. For example, a doctor treating an individual may configure the settings of an OSA stimulation system according to the disclosure based upon biomarker concentrations or amounts in a blood sample recently collected from the individual.

A system according to the disclosure may utilize one or more implanted sensors (e.g., incorporated into an implanted OSA stimulation system), without the need for sensor data collected from any external sensors. However, in some aspects, an implanted device may not have the sensors required to measure all of the parameters listed above. In such cases, one or more external sensors may be used to supplement the dataset available to the system, e.g., to include actigraphy, heart rate, blood oxygen level, blood pressure, EEG, single or low channel EEG, in-ear EEG, other brain activity like FNIRS (functional near infra-red spectroscopy), EMG, eye movement (EOG), and environmental data such a temperature, humidity, extraneous noise, etc. In that sense, it is understood that systems according to the disclosure are modular in the sense that the present systems may take into account sensor data collected using any combination of internal and external sensors, and may further be configured to take into account parameters based on environmental data (e.g., temperature) and biomarker concentrations or amounts (e.g., determined based upon an analysis of a subject's blood).

In some aspects, the present disclosure contemplates using data from an active implantable device with or without supplemental data from one or more external devices to help determine a sleep score, which is a measure of sleep quality for a person on a given night. The available data may be used as a proxy for sleep stage determination and duration, and quality. In some aspects, artificial intelligence, machine learning, and/or deep learning may be used to compare data collected from a patient with generalized data sets to develop or inform one or more classification algorithms. In another aspect, the data collected from a patient may be compared with data collected during a PSG for a single (or multiple) nights to inform/train the sleep stage and/or sleep quality classification algorithm for that patient. In another aspect, data collected from an implanted sensor and one or more external sensors, obtained from several patients, can be compared with PSG data from these patients to more broadly inform a population-based sleep stage and/or sleep quality classification algorithm.

In this manner, once a classifier algorithm has been trained, it can subsequently monitor sleep stage and hence sleep quality on a nightly basis for a person being treated using the systems described herein. The computation used to generate a classification, which is typically based on the data collected for that night (but which may alternately include multiple days and nights), could be performed by an implanted component of an OSA stimulation system (e.g., a controller within the housing of an implant), by an external controller that is able to communicate with the OSA stimulation system, by an application that is able to communicate with the OSA stimulation system, or remotely (e.g., by a cloud-based or remote server).

In some aspects, the systems described herein may be configured to generate a numeric sleep score as the primary output calculated, several domains or sub-domains may also be calculated as “sub-scores”), such as the percent of time spent in any sleep stage, ratios of time spent in certain sleep stages, number of awakenings, number of sleep-disordered breathing (“SDB”) events, correlations between certain measured parameters and sleep quality, etc. In some aspects, the sleep score and/or sub-scores may be used to motivate the patient to be more compliant with their therapy by illustrating how their sleep (and/or other physiological parameters) improves when they use the device. It may also illustrate how certain behaviors (e.g., activity, alcohol consumption, overeating, salt consumption, late night snacking, afternoon napping, etc.) can impact sleep quality to motivate better behavior or lifestyle choices. In some aspects, a patient's sleep scores, sub-scores, and individual measured parameters may be compared with other patients for whom data has been collected, in order to provide a rank or otherwise illustrate how they are similar or different to other people. This could be a comparison to all other patients, or patients that share one or more similar traits to the target patient (e.g., age, general health, blood pressure, degree of SDB, etc.). Awards, either virtual or tangible, could be given for improvements in behavior and/or scores.

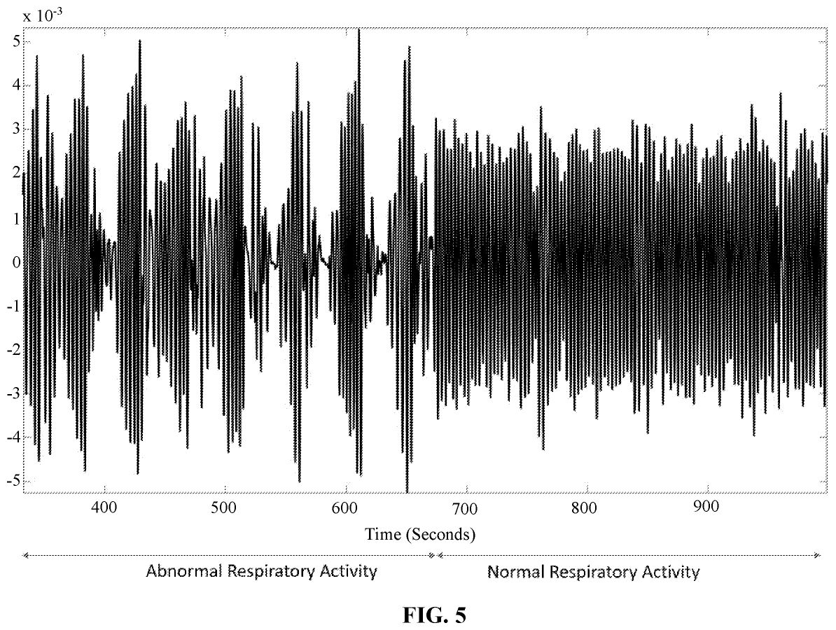

In some aspects, the systems described herein may be configured to detect SDB events (e.g., in connection with the generation of a sleep quality score, as described herein), by generating a respiratory waveform using data collected from one or more of the sensors described herein, and analyzing the respiratory waveform. During normal respiratory activity, the generated respiratory waveform will appear similar to a sinusoid with sustained amplitude. In contrast, during a respiratory event (abnormal respiratory activity), there is a suppression of chest activity followed by an oscillatory activity that corresponds to gasping of air to recover. The controller of the systems described herein may be configured to detect and/or classify respiratory events based upon this disruption in respiratory activity, e.g., a reduction in amplitude followed by oscillatory activity may be identified as a respiratory event. In some aspects, a controller may be configured to detect the amplitude of one or more peaks of the generated respiratory waveform (e.g., over a fixed or rolling window of time) in order to identify oscillatory activity. For example, if the peaks do not have a significant change in amplitude, then the signal may be classified as normal respiratory activity. In some aspects, a significant change may comprise a change of more than 20, 21, 22, 23, 24, 25, 26, 27, 28, 29, 30, 31, 32, 33, 34, 35, 36, 37, 38, 39, or 40%, or a change within a range bounded by any of the foregoing values, as compared to an average peak amplitude (e.g., for a window of time) or compared to a prior peak (e.g., the last peak detected, or a peak that occurred within 1, 2, 3, 4, or 5 seconds). If the amplitude of peaks decreases significantly (e.g., by 30%) and then continues to increases gradually (e.g., by 20, 21, 22, 23, 24, 25, 26, 27, 28, 29, 30, 31, 32, 33, 34, 35, 36, 37, 38, 39, or 40%, over the following 5, 6, 7, 8, 9, 10, 11, 12, 13, 14, or 15 peaks, that portion of the respiratory waveform may be classified as corresponding to abnormal respiratory activity. A respiratory waveform showing normal respiratory activity and abnormal respiratory activity in accordance with this aspect of the disclosure is provided as FIG. 5. This example illustrates a period of abnormal respiratory activity (left) followed by a period of normal respiratory activity (right). In many instances, such as the one shown in this example, abnormal activity may occur multiple times consecutively before normal breathing is restored. Accordingly, in some aspects the systems described herein may be configured to increase the amplitude of stimulation after the first detection of abnormal activity to help stimulate the subject out of a respiratory event.

In some aspects, a sleep quality score may be provided to a user via an interface of the system or using an electronic device communicatively-linked to the system (e.g., a mobile application executed on a smart phone, tablet, or external controller wirelessly paired with the system). In some aspects, the system may be configured to also provide recommendations to the patient using an interface or paired electronic device use (e.g., to remind the user to turn on the OSA stimulation system, or to change the system's settings), and to suggest behavior modification(s) (e.g., advising a patient not to eat after a particular time, or to adjust the temperature of the room where they intend to sleep). The systems described herein may also be configured to alert a physician when intervention may be needed (e.g., a device fault is detected, or reprogramming may be required) and to offer to set up an appointment (e.g., in person or via telemedicine) for the patient. In some aspects, a physician is able to interrogate and program the system, and any of the implanted or external sensors remotely. In some aspects, a software application (or controller or other device configured to control the systems described herein) may also engage the patient in other ways. For example, it may collect information provided by the patient (e.g., questionnaires, polls), and/or it could offer to facilitate a post to social media about the patient's status that day, or satisfaction with the device. It may also collect voice data, including testimonials or observations.

The systems described herein provide various benefits. For example, in some aspects such systems utilize sensors, or parameters imputed from sensors routinely found in implantable devices (especially active implantable devices) to determine a sleep stage and/or sleep score. In some aspects, the present systems leverages PSG study data to calibrate detection algorithms, train detection and classification algorithms, and/or as output data for machine learning or deep learning algorithms. The present systems may further be used to augment the data collected from an implanted sensor with sensor data from increasingly ubiquitous wearable devices, to further refine sleep stage determination and scoring.

As explained herein, the present systems may also be used in conjunction with a digital platform (e.g., local application(s) combined with edge and/or cloud-based processing and storage) to collect data from an implanted sensor (and optionally wearable devices), send the collected data to the cloud, compute sleep scores (and optionally sub-scores) locally or in the cloud, and share these insights with both the patient and a remote clinician. The sleep score, sub-scores, etc., can be used to motivate the patient to take certain actions, including potentially adjusting settings on their implant, actively choosing to use their implant more, schedule visits with their medical provider, participate in a poll, and post to social media sites. Patient motivation may be in the form of a competition, comparing their scores to other patients, or other patients that are similar to them in some way, providing a ranking and showing how their score and/or their ranking have improved over time because of actions they have taken.

Finally, sleep stage and/or quality determinations produced by the systems described herein may be used to alert clinicians to potential issues, and potentially adjust the patient's OSA stimulation system's settings remotely. In summary, the present systems provide multiple options for increasing patient engagement, therapy compliance, and the ease at which clinicians manage patients, thereby improving therapeutic outcomes.

Classifiers

The term “classifier,” as used herein, refers broadly to a machine learning algorithm such as support vector machine(s), AdaBoost classifier(s), penalized logistic regression, elastic nets, regression tree system(s), gradient tree boosting system(s), naive Bayes classifier(s), neural nets, Bayesian neural nets, k-nearest neighbor classifier(s), deep learning systems, and random forest classifiers. The systems and methods described may use any of these classifiers, or combinations thereof.

A “Classification and Regression Trees (CART),” as used herein, refers broadly to a method to create decision trees based on recursively partitioning a data space so as to optimize one or more metrics, e.g., model performance.

The classification systems used herein may include computer executable software, firmware, hardware, or combinations thereof. For example, the classification systems may include reference to a processor and supporting data storage. Further, the classification systems may be implemented across multiple devices or other components local or remote to one another. The classification systems may be implemented in a centralized system, or as a distributed system for additional scalability. Moreover, any reference to software may include non-transitory computer readable media that when executed on a computer, causes the computer to perform one or more steps.

There are many potential classifiers that can be used by the systems and methods described herein. Machine and deep learning classifiers include but are not limited to AdaBoost, Artificial Neural Network (“ANN”) learning algorithms, Bayesian belief networks, Bayesian classifiers, Bayesian neural networks, Boosted trees, case-based reasoning, classification trees, Convolutional Neural Networks, decisions trees, Deep Learning, elastic nets, Fully Convolutional Networks (FCN), genetic algorithms, gradient boosting trees, k-nearest neighbor classifiers, LASSO, Linear Classifiers, naive Bayes classifiers, neural nets, penalized logistic regression, Random Forests, ridge regression, support vector machines, or an ensemble thereof. See, e.g., Han & Kamber (2006) Chapter 6, Data Mining, Concepts and Techniques, 2nd Ed. Elsevier: Amsterdam. As described herein, any classifier or combination of classifiers (e.g., an ensemble) may be used by the present systems.

Deep Learning Algorithms

In some aspects, the classifier is a deep learning algorithm. Machine learning is a subset of artificial intelligence that uses a machine's ability to take a set of data and learn about the information it is processing by changing the algorithm as data is being processed. Deep learning is a subset of machine learning that often utilizes artificial neural networks inspired by the workings on the human brain. For example, the deep learning architecture may be multilayer perceptron neural network (“MLPNN”), backpropagation, Convolutional Neural Network (“CNN”), Recurrent Neural Network (“RNN”), Long Short-Term Memory (“LSTM”), Generative Adversarial Network (“GAN”), Restricted Boltzmann Machine (“RBM”), Deep Belief Network (“DBN”), or an ensemble thereof.

Classification Trees

A classification tree is an easily interpretable classifier with built in feature selection. A classification tree recursively splits the data space in such a way so as to maximize the proportion of observations from one class in each subspace.

The process of recursively splitting the data space creates a binary tree with a condition that is tested at each vertex. A new observation is classified by following the branches of the tree until a leaf is reached. At each leaf, a probability is assigned to the observation that it belongs to a given class. The class with the highest probability is the one to which the new observation is classified. Classification trees are essentially a decision tree whose attributes are framed in the language of statistics. They are highly flexible but very noisy (the variance of the error is large compared to other methods).

Tools for implementing classification tree are available, by way of non-limiting example, for the statistical software computing language and environment, R. For example, the R package “tree,” version 1.0-28, includes tools for creating, processing and utilizing classification trees. Examples of Classification Trees include but are not limited to Random Forest. See also Kaminski et al. (2017) “A framework for sensitivity analysis of decision trees.” Central European Journal of Operations Research. 26(1): 135-159; Karimi & Hamilton (2011) “Generation and Interpretation of Temporal Decision Rules”, International Journal of Computer Information Systems and Industrial Management Applications, Volume 3, the content of which is incorporated by reference in its entirety.

Random Forest Classifiers

Classification trees are typically noisy. Random forests attempt to reduce this noise by taking the average of many trees. The result is a classifier whose error has reduced variance compared to a classification tree. Methods of building a Random Forest classifier, including software, are known in the art. Prinzie & Poel (2007) “Random Multiclass Classification: Generalizing Random Forests to Random MNL and Random NB.” Database and Expert Systems Applications. Lecture Notes in Computer Science. 4653; Denisko & Hoffman (2018) “Classification and interaction in random forests.” PNAS 115(8): 1690-1692, the contents of which are incorporated by reference in its entirety.

To classify a new observation using the random forest, classify the new observation using each classification tree in the random forest. The class to which the new observation is classified most often amongst the classification trees is the class to which the random forest classifies the new observation. Random forests reduce many of the problems found in classification trees but at the tradeoff of interpretability.

Tools for implementing random forests as discussed herein are available, by way of non-limiting example, for the statistical software computing language and environment, R. For example, the R package “random Forest,” version 4.6-2, includes tools for creating, processing and utilizing random forests.

AdaBoost (Adaptive Boosting)

AdaBoost provides a way to classify each of n subjects into two or more categories based on one k-dimensional vector (called a k-tuple) of measurements per subject. AdaBoost takes a series of “weak” classifiers that have poor, though better than random, predictive performance and combines them to create a superior classifier. The weak classifiers that AdaBoost uses are classification and regression trees (“CARTs”). CARTs recursively partition the dataspace into regions in which all new observations that lie within that region are assigned a certain category label. AdaBoost builds a series of CARTs based on weighted versions of the dataset whose weights depend on the performance of the classifier at the previous iteration. See Han & Kamber (2006) Data Mining, Concepts and Techniques, 2nd Ed. Elsevier: Amsterdam, the content of which is incorporated by reference in its entirety. AdaBoost technically works only when there are two categories to which the observation can belong. For g>2 categories, (g/2) models must be created that classify observations as belonging to a group of not. The results from these models can then be combined to predict the group membership of the particular observation. Predictive performance in this context is defined as the proportion of observations misclassified.

Convolutional Neural Network

Convolutional Neural Networks (“CNNs” or “ConvNets”) are a class of deep, feed-forward artificial neural networks, most commonly applied to analyzing visual imagery. CNNs use a variation of multilayer perceptrons designed to require minimal preprocessing. They are also known as shift invariant or space invariant artificial neural networks (“SIANN”), based on their shared-weights architecture and translation invariance characteristics. Convolutional networks were inspired by biological processes in that the connectivity pattern between neurons resembles the organization of the animal visual cortex. Individual cortical neurons respond to stimuli only in a restricted region of the visual field known as the receptive field. The receptive fields of different neurons partially overlap such that they cover the entire visual field. CNNs use relatively little pre-processing compared to other image classification algorithms This means that the network learns the filters that in traditional algorithms were hand-engineered. This independence from prior knowledge and human effort in feature design is a major advantage. LeCun and Bengio (1995) “Convolutional networks for images, speech, and time-series,” in Arbib (Ed.), The Handbook of Brain Theory and Neural Networks, MIT Press, the content of which is incorporated by reference in its entirety. Fully convolutional indicates that the neural network is composed of convolutional layers without any fully connected layers or MLP usually found at the end of the network. A CNN is an example of deep learning.

Support Vector Machines

Support vector machines (“SVMs”) are recognized in the art. In general, SVMs provide a model for use in classifying each of n subjects to two or more categories based on one k-dimensional vector (called a k-tuple) per subject. An SVM first transforms the k-tuples using a kernel function into a space of equal or higher dimension. The kernel function projects the data into a space where the categories can be better separated using hyperplanes than would be possible in the original data space. To determine the hyperplanes with which to discriminate between categories, a set of support vectors, which lie closest to the boundary between the disease categories, may be chosen. A hyperplane is then selected by known SVM techniques such that the distance between the support vectors and the hyperplane is maximal within the bounds of a cost function that penalizes incorrect predictions. This hyperplane is the one which optimally separates the data in terms of prediction. Vapnik (1998) Statistical Learning Theory; Vapnik “An overview of statistical learning theory” IEEE Transactions on Neural Networks 10(5): 988-999 (1999) the content of which is incorporated by reference in its entirety. Any new observation is then classified as belonging to any one of the categories of interest, based where the observation lies in relation to the hyperplane. When more than two categories are considered, the process is carried out pairwise for all of the categories and those results combined to create a rule to discriminate between all the categories. See Cristianini, N., & Shawe-Taylor, J. (2000). An Introduction to Support Vector Machines and Other Kernel-based Learning Methods. Cambridge: Cambridge University Press provides some notation for support vector machines, as well as an overview of the method by which they discriminate between observations from multiple groups.

In an exemplary embodiment, a kernel function known as the Gaussian Radial Basis Function (RBF) is used. Vapnik, 1998. The RBF may be used when no a priori knowledge is available with which to choose from a number of other defined kernel functions such as the polynomial or sigmoid kernels. See Han et al. Data Mining: Concepts and Techniques, Morgan Kaufman 3rd Ed. (2012). The RBF projects the original space into a new space of infinite dimension. A discussion of this subject and its implementation in the R statistical language can be found in Karatzoglou et al. “Support Vector Machines in R,” Journal of Statistical Software 15(9) (2006), the content of which is incorporated by reference in its entirety. SVMs may be fitted using the ksvm( ) function in the kernlab package. Other suitable kernel functions include, but are not limited to, linear kernels, radial basis kernels, polynomial kernels, uniform kernels, triangle kernels, Epanechnikov kernels, quartic (biweight) kernels, tricube (triweight) kernels, and cosine kernels. Support vector machines are one out of many possible classifiers that could be used on the data. By way of non-limiting example, and as discussed below, other methods such as naive Bayes classifiers, classification trees, k-nearest neighbor classifiers, etc. may be used on the same data used to train and verify the support vector machine.

Naïve Bayes Classifier

The set of Bayes Classifiers are a set of classifiers based on Bayes' Theorem. See, e.g., Joyce (2003), Zalta, Edward N. (ed.), “Bayes' Theorem”, The Stanford Encyclopaedia of Philosophy (Spring 2019 Ed.), Metaphysics Research Lab, Stanford University, the content of which is incorporated by reference in its entirety.

Classifiers of this type seek to find the probability that an observation belongs to a class given the data for that observation. The class with the highest probability is the one to which each new observation is assigned. Theoretically, Bayes classifiers have the lowest error rates amongst the set of classifiers. In practice, this does not always occur due to violations of the assumptions made about the data when applying a Bayes classifier.

The naïve Bayes classifier is one example of a Bayes classifier. It simplifies the calculations of the probabilities used in classification by making the assumption that each class is independent of the other classes given the data. Naïve Bayes classifiers are used in many prominent anti-spam filters due to the ease of implantation and speed of classification but have the drawback that the assumptions required are rarely met in practice. Tools for implementing naive Bayes classifiers as discussed herein are available for the statistical software computing language and environment, R. For example, the R package “e1071,” version 1.5-25, includes tools for creating, processing, and utilizing naive Bayes classifiers.

Neural Networks

One way to think of a neural network is as a weighted directed graph where the edges and their weights represent the influence each vertex has on the others to which it is connected. There are two parts to a neural network: the input layer (formed by the data) and the output layer (the values, in this case classes, to be predicted). Between the input layer and the output layer is a network of hidden vertices. There may be, depending on the way the neural network is designed, several vertices between the input layer and the output layer.

Neural networks are widely used in artificial intelligence and data mining but there is the danger that the models the neural nets produce will over fit the data (i.e., the model will fit the current data very well but will not fit future data well). Tools for implementing neural nets as discussed herein are available for the statistical software computing language and environment, R. For example, the R package “e1071,” version 1.5-25, includes tools for creating, processing, and utilizing neural nets.

k-Nearest Neighbor Classifiers (KNN)

The nearest neighbor classifiers are a subset of memory-based classifiers. These are classifiers that have to “remember” what is in the training set in order to classify a new observation. Nearest neighbor classifiers do not require a model to be fit.

To create a k-nearest neighbor (knn) classifier, the following steps are taken:
1. Calculate the distance from the observation to be classified to each observation in the training set. The distance can be calculated using any valid metric, though Euclidian and Mahalanobis distances are often used. The Mahalanobis distance is a metric that takes into account the covariance between variables in the observations.
2. Count the number of observations amongst the k nearest observations that belong to each group.
3. The group that has the highest count is the group to which the new observation is as signed.

Nearest neighbor algorithms have problems dealing with categorical data due to the requirement that a distance be calculated between two points but that can be overcome by defining a distance arbitrarily between any two groups. This class of algorithm is also sensitive to changes in scale and metric. With these issues in mind, nearest neighbor algorithms can be very powerful, especially in large data sets. Tools for implementing k-nearest neighbor classifiers as discussed herein are available for the statistical software computing language and environment, R. For example, the R package “e1071,” version 1.5-25, includes tools for creating, processing, and utilizing k-nearest neighbor classifiers.

Training Data

In another aspect, methods described herein include training of about 75%, about 80%, about 85%, about 90%, or about 95% of the data in the library or database and testing the remaining percentage for a total of 100% data. In an aspect, from about 70% to about 90% of the data is trained and the remainder of about 10% to about 30% of the data is tested, from about 80% to about 95% of the data is trained and the remainder of about 5% to about 20% of the data is tested, or from about 90% of the data is trained and the remainder of about 10% of the data is tested.

In some aspects, the database or library contains data from the analysis of over about 25, about 60, over about 125, over about 250, over about 500, or over about 1000 human subjects (collected using systems according to the disclosure, PSG studies, etc.). In some aspects, the data may comprise data from healthy subjects and/or from those known to have OSA.

The training data may comprise, e.g., data relating to any of the parameters described herein, including sensor data, biomarker data, environmental data, or any combinations thereof.

Methods of Classification

The disclosure provides for methods of classifying data (e.g., sensor data and/or biomarker data) obtained from an individual in order to determine the individual's sleep stage and to generate a sleep quality score. In some aspects, these methods involve preparing or obtaining training data, as well as evaluating test data obtained from an individual (as compared to the training data), using one of the classification systems including at least one classifier as described above. Preferred classification systems use classifiers such as, but not limited to, support vector machines (SVM), AdaBoost, penalized logistic regression, naive Bayes classifiers, classification trees, k-nearest neighbor classifiers, Deep Learning classifiers, neural nets, random forests, Fully Convolutional Networks (FCN), Convolutional Neural Networks (CNN), and/or an ensemble thereof. Deep Learning classifiers are a more preferred classification system. The classification system may be configured, e.g., to output a determination as to a subject's sleep stage or a sleep quality score, based on sensor data, biomarker data, or combinations thereof.

As noted above, in some aspects a classifier may comprise an ensemble of multiple classifiers. For example, an ensemble method may include SVM, AdaBoost, penalized logistic regression, naive Bayes classifiers, classification trees, k-nearest neighbor classifiers, neural nets, Fully Convolutional Networks (FCN), Convolutional Neural Networks (CNN), Random Forests, deep learning, or any ensemble thereof, in order to make any of the determinations described herein.

An exemplary method for classifying sleep stage and/or quality may comprise the steps of: (a) accessing an electronically stored set of training data vectors, each training data vector or k-tuple representing an individual human subject and comprising sensor data and/or biometric data for the respective human subject for each replicate, the training data vector further comprising a classification with respect to a sleep stage and/or quality characterization of each respective human subject; (b) training an electronic representation of a classifier or an ensemble of classifiers as described herein using the electronically stored set of training data vectors; (c) receiving test data comprising a plurality of sensor data and/or biometric data for a test subject; (d) evaluating the test data using the electronic representation of the classifier and/or an ensemble of classifiers as described herein; and (e) outputting a classification of the test subject's sleep stage and/or quality based on the evaluating step. The test subject may be the same as the human subject used for training purposes (e.g., a baseline may be established for an individual using past data). In some aspects, the system will instead be trained with sensor data and/or biometric data obtained from a plurality of human subjects (e.g., a population which may contain healthy individuals known not to have OSA, individuals known to have OSA, or a combination thereof).

In another embodiment, the disclosure provides a method of classifying test data, the test data comprising sensor data and/or biometric data for a test subject, comprising: (a) accessing an electronically stored set of training data vectors, each training data vector or k-tuple representing an individual human subject and comprising sensor data and/or biometric data for the respective human subject for each replicate, the training data further comprising a classification with respect to sleep stage and/or sleep quality for the respective human subject; (b) using the electronically stored set of training data vectors to build a classifier and/or ensemble of classifiers; (c) receiving test data comprising a plurality of sensor data and/or biometric data for a human test subject; (d) evaluating the test data using the classifier(s); and (e) outputting a classification as to the sleep stage and/or sleep quality of the human test subject based on the evaluating step. Alternatively, all (or any combination of) the replicates may be averaged to produce a single value. Outputting in accordance with this invention includes displaying information regarding the classification of the human test subject in an electronic display in human-readable form. The sensor data and/or biometric data may comprise data in accordance with any of the exemplary aspects of the present systems and methods described herein. In some aspects, the set of training vectors may comprise at least 20, 25, 30, 35, 50, 75, 100, 125, 150, or more vectors.

Classifier-Based Systems and Methods

As explained above, the systems and methods provided herein may be used to determine (and/or monitor) a human subject's sleep stage and/or sleep quality, and optionally to treat OSA. OSA stimulation systems according to the disclosure possess several advantages compared to prior systems, and in particular allow for more accurate tailoring of stimulation-based parameters, and power savings (e.g., components of the OSA stimulation system may be disabled or switched to a low-power mode when a subject is found to be awake or in a sleep stage wherein stimulation is reduced or unnecessary). Moreover, the present systems are advantageous in that they do not require invasive or uncomfortable sensors, improving the likelihood of patient compliance and positive therapeutic outcomes.

Prior systems based on EEG and electrooculography (“EOG”) provide a reliable way to detect wakefulness and sleep stage. However, such systems require many wires and instrumentation that can interfere with sleep. In contrast, OSA stimulation systems according to the disclosure may are able to detect sleep stage and wakefulness in order to be able to automatically start and stop (or otherwise modulate) treatment and to determine sleep quality. The paper “Respiratory rate variability in sleeping adults without obstructive sleep apnea” (G. Gutierrez et al, Physiol. Rep., 4:17, 2016) (hereinafter, “Guierrez 2016”) describes an approach for using nasal cannula pressure respiration rate variability to determine wakefulness. However, this approach requires the use of a nasal cannula or nasal thermistor, which can also impede sleep. Moreover, the technique described in Gutierrez 2016 requires computation of fast Fourier transformations (“FFTs”, a processor-intensive calculation) and only produce a sleep stage prediction every 2.7 seconds.

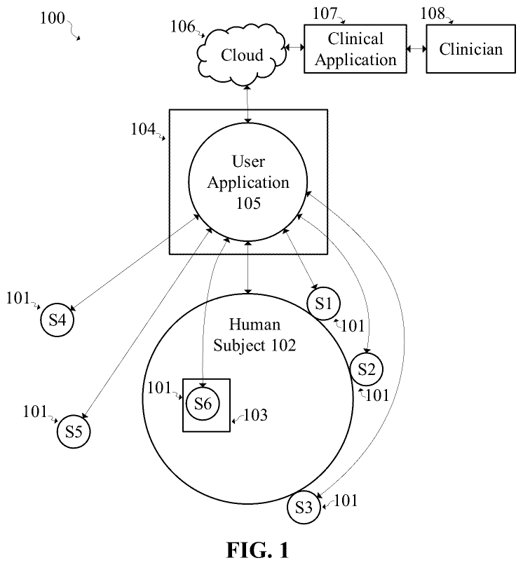

FIG. 1 is a diagram illustrating an exemplary embodiment of a system for treating OSA (100) using sensor data obtained from a combination of implanted and external sensors (labeled S1-S6 (101). In this example, the system comprises five external sensors (101), labeled S1-S5, which may be placed on or in proximity to a human subject being treated (e.g., via an adhesive or strap) and an implanted sensor S6 (101), which is integrated into an implanted OSA stimulation system 103. This implanted sensor, and the five external sensors, are communicatively linked to a user application 105 executed on a controller 104. In this example, the controller is shown to be located external to the user and may, e.g., comprise a component of a user device such as a tablet, smartphone, or dedicated external controller for the OSA stimulation system 103. In other aspects, the controller may comprise an implanted component of an OSA stimulation system 103 (e.g., a controller, and optionally one or more sensors, may be integrated into an implanted housing). It is understood that the controller may be located in any housing of an OSA stimulation system, as an external device, or as a separate implant, in various aspects. In this case, the user application 105 is configured to communicate with a clinical application 107 via an intervening cloud infrastructure 106, allowing a remote clinician 108 to interact with the controller 104. This configuration may allow for a clinician 108 to view a user's sleep score and/or sleep quality determinations, to view sensor data and/or biometric data, and to view and/or modify one or more settings of the OSA stimulation system.

In this exemplary embodiment, the implanted OSA stimulation system comprises a housing (103) that includes both an implantable pulse generator (“IPG”), at least one implanted sensor S6 (101) and a controller configured to handle signal processing and storage, operation of the OSA stimulation system, and wireless communication between the OSA stimulation system 103 and the user application 105 executed on the external controller 104. The OSA stimulation 103 system further includes one or more electrodes to deliver stimulation to one or more nerves which innervate an upper airway muscle of the human subject. As described herein, the system (100) may be used to treat OSA based upon the subject's sleep stage and/or sleep quality, which may be determined by the controller 104 (or by the clinical application 107) using any of the techniques described herein. In alternative aspects, the determination may be performed by an implanted controller (e.g., of an OSA stimulation system). However, in some aspects power and processing resources may be leveraged more efficiently when this determination is offloaded to an external controller (e.g., an external electronic device, whether local, remote or cloud-based).

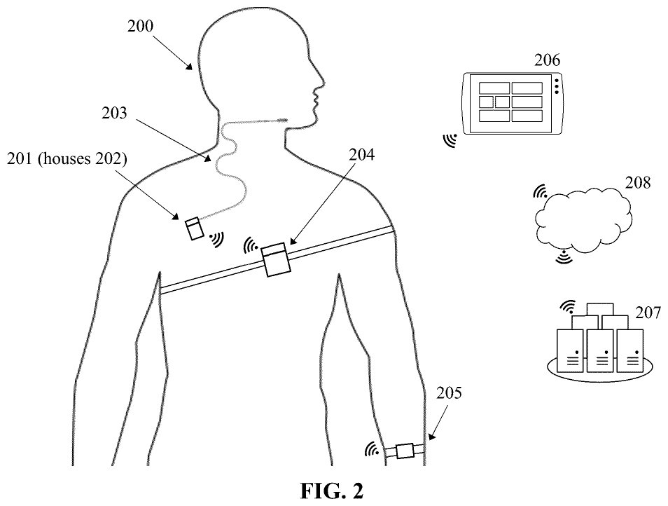

FIG. 2 is a diagram showing another exemplary system 200 according to the disclosure. In this example, an implanted OSA stimulation system 201 includes an implanted controller 202 within the same housing as used for the OSA stimulation system 201. One or more electrodes extend from the housing of the OSA stimulation system 201 to deliver stimulation to one or more nerves which innervate an upper airway muscle of the human subject. In this case, the implanted controller 202 is configured to communicate wirelessly with one or more external sensors, such as the pressure sensor 204 shown affixed to the user's chest via a strap or harness, and the heart rate sensor provided in a wrist-worn device 205. The implanted controller 202 may further communicate wirelessly with an external controller 206 (e.g., a discrete controller with a user interface for viewing and/or modifying settings of the OSA stimulation system, and for viewing sleep stage and/or quality determinations and collected sensor/biomarker data). In this case, the external controller 206 is configured to communicate with a remote server 207 via cloud-based infrastructure 208.

This exemplary configuration allows for sensor data and/or biomarker data to be stored and/or processed locally (e.g., by the implanted controller 202 or external controller 206), or remotely by the remote server 207. Accordingly, processing may be shifted as needed based on power or processing constraints. For example, in some aspects a local implanted or external controller may be configured to perform low-intensity processing, and/or periodic processing, to minimize power requirements, whereas more complicated processing may be performed by an external server. Some machine learning and artificial intelligence-based algorithms require significant processing and power resources. As such, it may be efficient to direct such processing to a remote server capable of efficiently handling the necessary calculations.

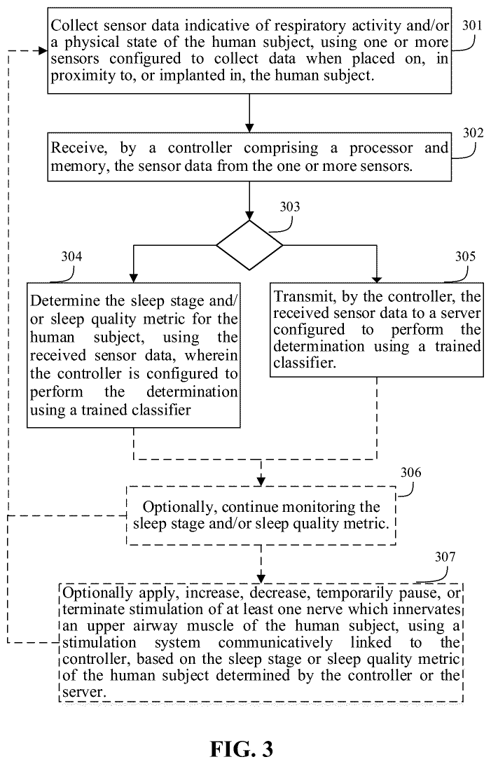

FIG. 3 is a conceptual flow diagram summarizing a method for determining a subject's sleep stage and/or sleep quality. As shown by this figure, the process may begin with the collection of sensor data indicative of respiratory activity and/or a physical state of the human subject, using one or more sensors configured to collect data when placed on, in proximity to, or implanted in, the human subject (301). This sensor data may be transmitted to a controller comprising a processor and memory (302). At the next step, the process may bifurcate depending on the needs of a given application. In some embodiments, the controller may be configured to determine the sleep stage and/or sleep quality metric for the human subject, using the received sensor data, wherein the controller is configured to perform the determination using a trained classifier (304). In others, the controller may be configured to transmit the received sensor data to a server configured to perform the determination using a trained classifier (305).

At this point, the system may optionally continue to monitor the sleep stage and/or sleep quality of the human subject (306), e.g., throughout the course of a night. Moreover, the system may optionally be configured to apply, increase, decrease, temporarily pause, or terminate stimulation of at least one nerve which innervates an upper airway muscle of the human subject, using a stimulation system communicatively linked to the controller, based on the sleep stage or sleep quality metric of the human subject determined by the controller or the server (307).

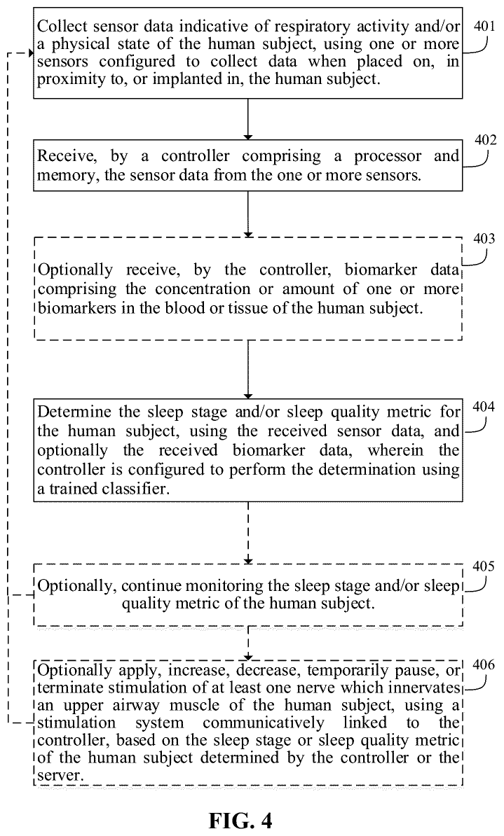

FIG. 4 is a conceptual flow diagram showing another exemplary method for determining a sleep stage and/or sleep quality metric for a human subject. As illustrated by this figure, such methods may optionally integrate the use of biomarker data for the human subject (e.g., the concentration of one or more biomarkers, such as a subject's glucose level). This exemplary method begins with the collection of sensor data indicative of respiratory activity and/or a physical state of the human subject, using one or more sensors configured to collect data when placed on, in proximity to, or implanted in, the human subject (401), which is in turn transmitted to a controller comprising a processor and memory (402). The controller is configured to optionally receive biomarker data comprising the concentration or amount of one or more biomarkers in the blood or tissue of the human subject (403), and to determine the sleep stage and/or sleep quality metric for the human subject, using the received sensor data, and the received biomarker data (if provided), using a trained classifier (404). This example ends with optional monitoring (405) and treatment (406) steps, analogous to those discussed above in the context of FIG. 3.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular compound, composition, article, apparatus, methodology, protocol, and/or reagent, etc., described herein, unless expressly stated as such. In addition, those of ordinary skill in the art will recognize that certain changes, modifications, permutations, alterations, additions, subtractions and sub-combinations thereof can be made in accordance with the teachings herein without departing from the spirit of the present specification. It is therefore intended that the following appended claims and claims hereafter introduced are interpreted to include all such changes, modifications, permutations, alterations, additions, subtractions and sub-combinations as are within their true spirit and scope.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (and equivalent open-ended transitional phrases thereof like including, containing and having) encompasses all the expressly recited elements, limitations, steps and/or features alone or in combination with unrecited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amended for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such embodiments described herein or so claimed with the phrase “comprising” are expressly or inherently unambiguously described, enabled and supported herein for the phrases “consisting essentially of” and “consisting of”

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Lastly, the terminology used herein is for the purpose of describing particular embodiments only, and is not intended to limit the scope of the present invention, which is defined solely by the claims. Accordingly, the present invention is not limited to that precisely as shown and described.

What is claimed is:

1. A computer-implemented system for determining a sleep stage and/or a sleep quality metric for a human subject, comprising:
one or more sensors, wherein each sensor is configured to collect sensor data indicative of respiratory activity and/or a physical state of the human subject when placed on, in proximity to, or implanted in, the human subject; and
a controller comprising a processor and memory, communicatively linked to the one or more sensors and configured to
receive the sensor data from the one or more sensors, and
determine the sleep stage and/or sleep quality metric for the human subject, using the received sensor data,
wherein the controller is configured to perform the determination using a trained classifier comprising an electronic representation of a classification system.

2. The system of claim 1, wherein the one or more sensors each comprise: a pressure sensor, an accelerometer, a gyroscope, an auscultation sensor, a heart rate monitor, an electrocardiogram (“ECG”) sensor, a blood pressure sensor, a blood oxygen level sensor, an electromyography (“EMG”) sensor, and/or a muscle sympathetic nerve activity (“MSNA”) sensor.

3. The system of claim 1, wherein each sensor is independently positioned on, in proximity to, or as an implant within, the human subject.

4. The system of claim 1, wherein the controller is further configured to receive biomarker data for the human subject comprising a concentration or amount of one or more biomarkers of the human subject, and to use this biomarker data when determining the sleep stage and/or sleep quality metric for the human subject; optionally
wherein the biomarker data was generated from an assay of one or more biological fluid or tissue samples obtained from the human subject.

5. The system of claim 4, wherein the one or more biomarkers comprise a concentration or amount of epinephrine, norepinephrine, cortisol, melatonin, serotonin, glucose, insulin, dopamine, noradrenaline, 5-hydroxoindiolacytic acid, glutamate, blood alcohol, tryptophan, kynurenine, and/or one or more inflammatory cytokines, in the human subject's blood or tissue.

6. The system of claim 1, wherein the controller is configured to determine the sleep stage and/or sleep quality metric for the human subject using
a) sensor data received from at least or exactly 1, 2, 3, 4, 5, 6, 7, 8, 9, or 10 sensors; and/or
b) biomarker data comprising a concentration or amount of at least or exactly 1, 2, 3, 4, 5, 6, 7, 8, 9, or 10 biomarkers.

7. The system of claim 1, wherein the trained classifier was trained using a baseline dataset, wherein the baseline dataset comprises:
a) data generated during a prior single or multi-night polysomnography (“PSG”) study of the human subject; and/or
b) data generated from a prior single or multi-night PSG study of a population of human subjects.

8. The system of claim 7, wherein the baseline dataset comprises:
a) sensor data from one or more sensors, where each sensor comprises: a pressure sensor, an accelerometer, a gyroscope, an auscultation sensor, a heart rate monitor, an ECG sensor, a blood pressure sensor, a blood oxygen level sensor, an EMG sensor, and/or an MSNA sensor; and/or
b) concentration or amounts of one or more biomarkers comprise epinephrine, norepinephrine, cortisol, melatonin, serotonin, glucose, insulin, dopamine, noradrenaline, 5-hydroxoindiolacytic acid, glutamate, blood alcohol, tryptophan, kynurenine, and/or one or more inflammatory cytokines.

9. The system of claim 1, wherein the classifier comprises a machine learning and/or deep learning algorithm.

10. The system of claim 1, wherein the one or more sensors configured to collect sensor data indicative of respiratory activity and/or a physical state of the human subject does not include an electroencephalography (“EEG”) sensor.

11. The system of claim 1, wherein the sleep stage for the human subject is determined to be a sleep stage selected from awake, or N1, N2, N3, or REM sleep.

12. The system of claim 1, wherein the sleep quality metric for the human subject is determined to be a numeric score.

13. The system of claim 1, wherein the system is configured to output the determined sleep stage and/or sleep quality metric to a graphical or text-based interface of an electronic device.

14. The system of claim 13, wherein the electronic device is a discrete controller of the system, a computer, a smart phone, a tablet, or a wearable device.

15. A method for determining a sleep stage and/or a sleep quality metric for a human subject comprising:
collecting sensor data indicative of respiratory activity and/or a physical state of the human subject, using one or more sensors configured to collect data when placed on, in proximity to, or implanted in, the human subject;
receiving, by a controller comprising a processor and memory, the sensor data from the one or more sensors;
determining the sleep stage and/or sleep quality metric for the human subject, using the received sensor data;
wherein the controller is configured to
perform the determination using a trained classifier comprising an electronic representation of a classification system, and/or to
transmit the received sensor data to a server configured to perform the determination using a trained classifier comprising an electronic representation of a classification system.

16. A method for determining a sleep stage and/or a sleep quality metric for a human subject comprising:
providing the system of claim 1, and
determining the sleep stage and/or sleep quality metric for the human subject, using the provided system.

17. A computer-implemented system for determining a sleep stage and/or a sleep quality metric for a human subject, comprising:
one or more sensors, wherein each sensor is configured to collect sensor data indicative of respiratory activity and/or a physical state of the human subject when placed on, in proximity to, or implanted in, the human subject; and
a controller comprising a processor and memory, communicatively linked to the one or more sensors and configured to
receive the sensor data from the one or more sensors, and
transmit data based on the received sensor data to at least one local, remote, or cloud-based server,
wherein the at least one local, remote, or cloud-based server is configured to determine a sleep quality metric for the human subject using a trained classifier configured to process the transmitted data.

18. The system of claim 17, wherein the controller is further configured to transmit biomarker data for the human subject, comprising a concentration or amount of one or more biomarkers, to the at least one local, remote, or cloud-based server; and
the at least one local, remote, or cloud-based server is configured to use the transmitted biomarker data when determining the sleep quality metric for the human subject using the trained classifier.

19. A system for treating obstructive sleep apnea (“OSA”), comprising:
the system for determining a sleep stage and/or a sleep quality metric for a human subject, of claim 1, and
a stimulation system, communicatively linked to the controller and configured to deliver stimulation to a nerve which innervates an upper airway muscle of the human subject based on the sleep stage and/or sleep quality metric of the human subject determined by the controller.

20. The system for treating OSA of claim 19, wherein the controller is configured to cause the stimulation system to apply, increase, decrease, temporarily pause, or terminate the stimulation based on the sleep stage and/or sleep quality metric of the human subject.

21. The system for treating OSA of claim 19, wherein the controller is configured to cause the stimulation system to change an amplitude, pulse width, or frequency of the stimulation based on the sleep stage and/or sleep quality metric of the human subject.
